(19) United States
(12) Patent Application Publication
(10) Pub. No.: US 2024/0115752 A1
(43) Pub. Date: Apr. 11, 2024

(54) WINDOW AND DISPLAY DEVICE HAVING THE SAME

(71) Applicant: Samsung Display Co., Ltd., Yongin-si (KR)

(72) Inventors: SUNGWOO KIM, Yongin-si (KR); JEONG SEOK LEE, Yongin-si (KR); HYANGSHIN JEONG, Yongin-si (KR)

(21) Appl. No.: 18/448,826

(22) Filed: Aug. 11, 2023

(30) Foreign Application Priority Data
Oct. 7, 2022 (KR) 10-2022-0128398

(51) Int. Cl. A61L 9/12

(52) U.S. Cl. CPC A61L 9/12; G02B 1/18

(57) ABSTRACT

A window includes: a base layer; and a fragrance-emitting layer on the base layer and including a plurality of fragrance-emitting capsules; and an anti-fingerprint layer on the fragrance-emitting layer.

### CROSS-REFERENCE TO RELATED APPLICATION

The present application claims priority to and the benefit of Korean Patent Application No. 10-2022-0128398, filed on Oct. 7, 2022, in the Korean Intellectual Property Office, the entire content of which is incorporated herein by reference.

### BACKGROUND

### 1. Field

Aspects of some embodiments of the present disclosure herein relate to a window, and a display device having the same.

### 2. Description of the Related Art

A display device generally provides or displays information to a user by displaying various images on a display screen. For example, the display device generally provides or displays images generated by a display panel to a user through the window. The window may include one or more areas that transmit images and one or more areas that do not transmit images. The areas that do not transmit the images may include a bezel pattern, and may thus may prevent or reduce instances of the internal configuration of the display device being viewed by the user, and may also improve the aesthetic properties of the display device.

The above information disclosed in this Background section is only for enhancement of understanding of the background and therefore the information discussed in this Background section does not necessarily constitute prior art.

### SUMMARY

Aspects of some embodiments of the present disclosure include a window and display device with relatively improved reliability and aesthetic properties.

Aspects of some embodiments of the present disclosure may also include a window and a display device providing fragrance.

Aspects of some embodiments of the inventive concept include a window including a base layer, a fragrance-emitting layer on the base layer and including a plurality of fragrance-emitting capsules, and an anti-fingerprint layer on the fragrance-emitting layer.

According to some embodiments, the fragrance-emitting capsule may include a fragrance source, and a shell surrounding the fragrance source.

According to some embodiments, the fragrance source may be released from the shell by pressure applied to an upper surface of the window, and a fragrance lifespan being a period during which each of the fragrance source is released from each of the shell in about 90% to about 100% among the plurality of fragrance-emitting capsules may be about one month to about two years.

According to some embodiments, the fragrance source may include at least one of one of terpenes, terpenoids, phenylpropanoid, allyl isothiocyanate, or allicin.

According to some embodiments, the shell may include at least one of chitosan, cellulose, or melamine.

According to some embodiments, the fragrance-emitting layer may further include a base resin in which the fragrance-emitting capsules are dispersed. The base resin may combine the fragrance-emitting capsule and the base layer.

According to some embodiments of the inventive concept, the window may further include a first bonding layer between the fragrance-emitting layer and the anti-fingerprint layer and configured to bond the fragrance-emitting layer and the anti-fingerprint layer.

According to some embodiments, the first bonding layer may include a silicon oxide or an aluminum oxide.

According to some embodiments, the window may further include the anti-reflection layer between the fragrance-emitting layer and the anti-fingerprint layer.

According to some embodiments, the window may further include a second bonding layer between the anti-fingerprint layer and the anti-reflection layer and configured to bond the anti-fingerprint layer and the anti-reflection layer.

According to some embodiments, the window may further include an optical layer between the base layer and the fragrance-emitting layer, and a plurality of irregularities may be defined on an upper surface of the optical layer.

According to some embodiments, the window may have a refractive index of about 1.2 to about 1.8, have a surface roughness of about 1 μm to about 20 μm, and have a light transmittance of about 88% to about 100% for light in the wavelength range of about 400 nm to about 700.

According to some embodiments, the fragrance-emitting layer may have a thickness of about 1 nm to about 3000 nm.

According to some embodiments of the inventive concept, a display device includes a set module, a display module on the set module, and a window on the display module and the window includes a base layer on the display module, a fragrance-emitting layer on the base layer and including a plurality of fragrance-emitting capsules, and an anti-fingerprint layer on the fragrance-emitting layer.

According to some embodiments, the window may include a bezel region and a transmissive region, and the fragrance-emitting layer may overlap the bezel layer. According to some embodiments, the fragrance-emitting layer may not overlap the transmissive region.

According to some embodiments, the bezel region may include a first region and a second region, at least a portion of the display module may overlap the first region, at least a portion of the set module may overlap the second region, and the fragrance-emitting layer may overlap the second region.

### BRIEF DESCRIPTION OF THE DRAWINGS

The accompanying drawings are included to provide a further understanding of embodiments according to the inventive concept, and are incorporated in and constitute a part of this specification. The drawings illustrate embodiments of the inventive concept and, together with the description, serve to explain principles of the inventive concept. In the drawings:

FIG. 1 is a combined perspective view of a display device according to some embodiments;

FIG. 2 is an exploded perspective view of a display device according to some embodiments;

FIG. 3 is a cross-sectional view of a display module according to some embodiments;

FIG. 4 is a plan view of a window according to some embodiments;

FIG. 5A is a cross-sectional view taken along the line II-II′ of FIG. 4 according to some embodiments;

FIG. 5B is a cross-sectional view taken along the line I-I′ of FIG. 1 according to some embodiments;

FIG. 6 is a cross-sectional view illustrating some components included in a window according to some embodiments;

FIG. 7 is a diagram schematically illustrating a structure of a fragrance-emitting capsule according to some embodiments;

FIGS. 8A through 8C are each a cross-sectional view illustrating some components included in a window functional layer according to some embodiments; and

FIGS. 9A through 9D are each a cross-sectional view illustrating some components included in a window according to some embodiments.

### DETAILED DESCRIPTION

In embodiments according to the inventive concept, various modifications may be made, and various forms may be applied, and aspects of some embodiments will be illustrated in the drawings and described in more detail in the text. However, this is not intended to limit the inventive concept to a specific disclosure form, it should be understood to include all changes, equivalents, and substitutes included in the spirit and scope of the inventive concept.

It will be understood that when an element (or a region, a layer, a part) is referred to as being “on”, “connected to” or “coupled to” another element, it can be directly on, connected, or coupled to the other element or intervening elements may be present therebetween.

Like numbers refer to like elements throughout. The thickness and the ratio and the dimension of the element are exaggerated for effective description of the technical contents.

As used herein, the term “and/or” includes any and all combinations of one or more of the associated listed items.

It will be understood that, although the terms first, second, etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, layer or section without departing from the scope of the inventive concept. Similarly, a second element, component, region, layer or section may be termed a first element, component, region, layer or section. As used herein, the singular forms, “a”, “an” and “the” are intended to include the plural forms as well, unless the context clearly indicates otherwise.

Also, terms of “below”, “on lower side”, “above”, “on upper side”, or the like may be used to describe the relationships of the elements illustrated in the drawings. These terms have relative concepts and are described on the basis of the directions indicated in the drawings.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

It will be further understood that the terms “includes” and/or “including”, when used in this specification, specify the presence of stated features, integers, steps, operations, elements, and/or components, but do not preclude the presence or addition of one or more other features, integers, steps, operations, elements, components, and/or groups thereof.

Hereinafter, a cover window, a display device having the cover window, and a manufacturing method thereof will be explained in more detail with reference to the accompanying drawings.

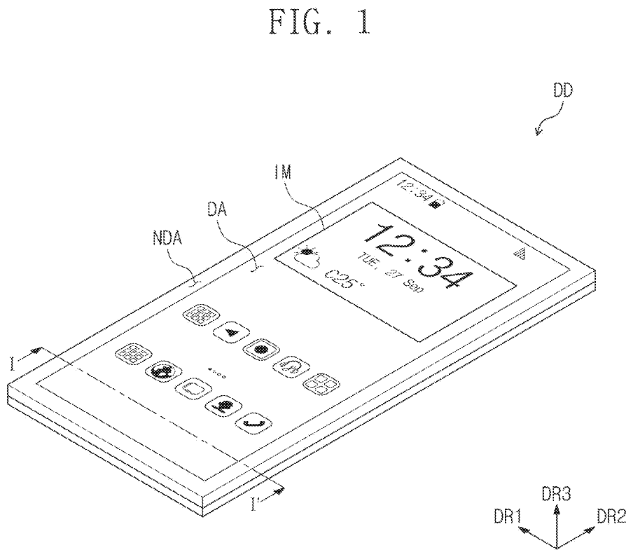

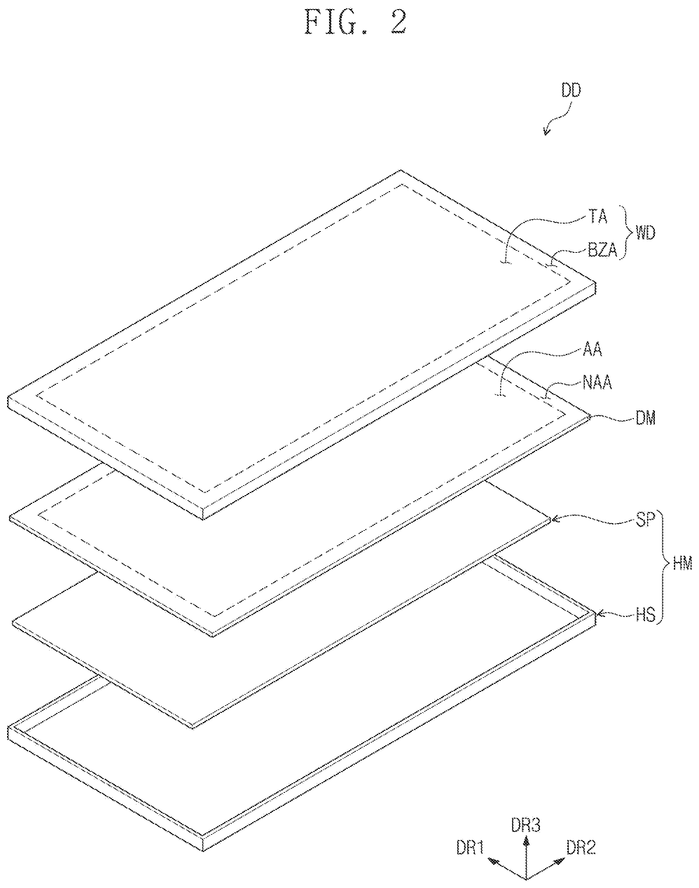

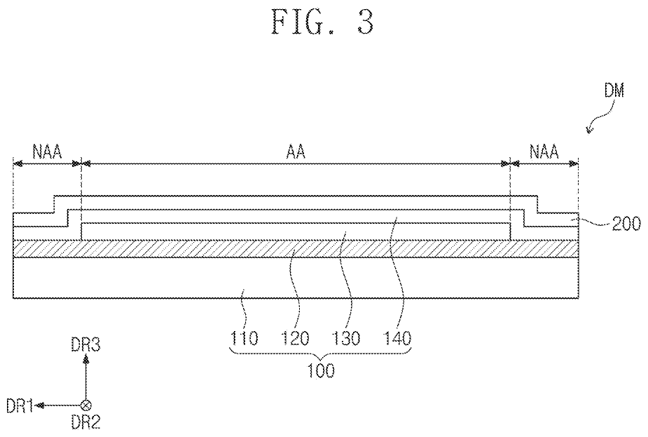

FIGS. 1 and 2 are perspective views illustrating a display device DD according to some embodiments. FIG. 3 is a cross-sectional view of a display module DM according to some embodiments.

FIG. 1 is a combined perspective view of a display device DD, and FIG. 2 is an exploded perspective view of the display device DD.

Referring to FIGS. 1 and 2, the display device DD may be a device activated in response to an electrical signal. For example, the display device DD may be a personal digital terminal, a tablet computer, a car navigation unit, a game console, or a wearable device, but embodiments of the inventive concept are not limited thereto. FIGS. 1 and 2 illustrate that the display device DD is a portable electronic device.

The display DD may display an image IM through a display region DA. The display region DA may include a plane defined by a first direction DR1 and a second direction DR2. However, a shape of the display region DA is not limited thereto, and for example, the display region DA may further include a curved surface bent from at least one side of the plane defined by the first direction DR1 and the second direction DR2.

A non-display region NDA is adjacent to the display region DA. The non-display region NDA may surround the display region DA. Accordingly, the shape of the display region DA may be defined substantially by the non-display region NDA. However, this is illustrated as an example, and the non-display region NDA may be located adjacent to only one side of the display region DA, or may be located in a transmissive region TA.

Meanwhile, FIG. 1 and the following drawings illustrate a first direction DR1 through a third direction DR3, and directions indicated by the first through the third direction DR1, DR2, and DR3 described herein may be relative concepts, and be thus changed into other directions. In this specification, the first direction DR1 and the second direction DR2 may be orthogonal to each other, and the third direction DR3 may be a normal direction of the plane defined by the first direction DR1 and the second direction DR2.

A thickness direction of the display device DD may be parallel to the third direction DR3, which is the normal direction of the plane defined by the first direction DR1 and the second direction DR2. In this specification, a front surface (or upper surface) and a rear surface (or lower surface) of each member constituting the display device DD may be defined with respect to the third direction DR3.

Referring to FIG. 2, the display device DD according to some embodiments may include a window WD, a display module DM, and a set module HM.

The window WD may be located on the display module DM and cover an entire outer side of the display module DM.

The window WD may have a shape corresponding to the shape of the display module DM. In display device DD according to some embodiments, the window WD may include an optically transparent insulating material. The window WD may include glass, sapphire, or polymer. For example, the window WD may include tempered glass that has been chemically strengthened.

The window WD may be divided into a transmissive region TA and a bezel region BZA. The transmissive region TA may be a part corresponding to the display region DA of the display device DD, and the bezel region BZA may be a part corresponding to the non-display region NDA of the display device DD. The bezel region BZA may define a shape of the transmissive region TA. The bezel region BZA may be adjacent to the transmissive region TA and surround the transmissive region TA. However, embodiments of the inventive concept are not limited thereto, and the bezel region BZA may be located adjacent to only one side of the transmissive region TA or be located within the transmissive region TA.

The display module DM may be located below the window WD. The display module DM may be a component, which generates or displays images and detects an input applied from the outside.

Referring to FIG. 3, the display module DM according to some embodiments may include a display panel 100 and a sensor layer 200.

The display panel 100 may be a component that substantially generates images. The display panel 100 may be a light-emitting-type display panel. For example, the display panel 100 may be an organic light-emitting display panel, an inorganic light-emitting display panel, a micro LED display panel, or a nano LED display panel. The display panel 100 may also be referred to as a display layer.

The display panel 100 may include a base substrate 110, a circuit layer 120, a light-emitting element layer 130, and an encapsulation layer 140.

The base substrate 110 may be a member providing a base substrate on which the circuit layer 120 is located. The base substrate 110 may be a rigid substrate or a flexible substrate capable of bending, folding, rolling, or the like. The base substrate 110 may be a glass substrate, a metal substrate, or a polymer substrate. However, embodiments of the inventive concept are not limited thereto, and the base substrate 110 may be an inorganic layer, an organic layer, or a composite material layer.

The base substrate 110 may have a multilayer structure. For example, the base substrate 110 may include a first synthetic resin layer, an inorganic layer having a single- or multi-layer structure, and a second synthetic resin layer located on the inorganic layer having a single- or multi-layer structure. Each of the first and second synthetic resin layers may include a polyimide-based resin, but embodiments of the inventive concept are not particularly limited thereto.

The circuit layer 120 may be located on the base substrate 110. The circuit layer 120 may include an insulating layer, a semiconductor pattern, a conductive pattern, a signal line, and the like. The insulating layer, a semiconductive layer, and a conductive layer may be formed through coating, depositing, etc., and the insulating layer, the semiconductor layer, and the conductive layer may be selectively patterned by performing a photolithography process and an etching process multiple times.

The light-emitting element layer 130 may be located on the circuit layer 120. The light-emitting element layer 130 may include a light-emitting element. For example, the light-emitting element may include an organic light-emitting material, an inorganic light-emitting material, an organic-inorganic light-emitting material, a quantum dot, a quantum rod, a micro LED, or a nano LED.

The encapsulation layer 140 may be located on the light-emitting element layer 130. The encapsulation layer 140 may protect the light-emitting element layer 130 from foreign substances such as moisture, oxygen, and dust particles. The encapsulation layer 140 may include at least one inorganic layer. The encapsulation layer 140 may include a stacked structure of inorganic layer/organic layer/inorganic layer.

The sensor layer 200 may be located on the display panel 100. The sensor layer 200 may detect an external input applied from the outside. The external input may be a user's input. The user's input may include various types of external inputs such as a touch by the user's body part, light, heat, pen, pressure, or the like.

The sensor layer 200 may be formed on the display panel 100 through a continuous process. In this case, the sensor layer 200 may be directly located on the display panel 100. Herein, “directly located” may mean that a third component is not located between the sensor layer 200 and the display panel 100. That is, an additional adhesive member may not be located between the sensor layer 200 and the display panel 100.

The anti-reflection layer may be further located on the sensor layer 200. The anti-reflection layer may reduce the reflectance for external light incident from the outside of the display device DD. The anti-reflection layer may be formed on the sensor layer 200 through a continuous process. The anti-reflection layer may include color filters. The color filters may have an arrangement (e.g., a set or predetermined arrangement). For example, the color filters may be arranged in consideration of colors of light emitted from pixels included in the display panel 100. In addition, the anti-reflection layer may further include a black matrix adjacent to the color filters.

According to some embodiments of the inventive concept, the sensor layer 200 may be omitted.

According to some embodiments, the display module DM may further include an adhesive layer. The display module DM may be attached to the window WD by the adhesive layer. The adhesive layer may have transparency, and may be, for example, an optically clear adhesive (OCA).

In the display module DM, an active region AA and a peripheral region NAA may be defined. The active region AA may be a region activated in response to an electrical signal. A driving circuit, a driving line or the like for driving the active region AA may be located in the peripheral region NAA.

The active region AA may correspond to the display region DA of the display device DD illustrated in FIG. 1. The peripheral region NAA may correspond to the non-display region NDA of the display device DD illustrated in FIG. 1.

The peripheral region NAA may be arranged to surround the active region AA. However, embodiments of the inventive concept are not limited thereto. According to some embodiments, the peripheral region NAA may be located adjacent to only one side of the active region AA, or be located in the active region AA.

The set module HM may include a support layer SP and a housing HS. The support layer SP may have a single-layer structure or a multilayer structure. The support layer SP may support the display module DM and the window WD. For example, the support layer SP may include at least one of a metal layer, a cushion layer, or a heat dissipation layer.

The housing HS may accommodate the display module DM and the support layer SP, and may be coupled to the window WD. The housing HS may be formed by assembling a plurality of parts, or formed from a one-body that has been injection-molded. The housing HS may include glass, plastic, or metal.

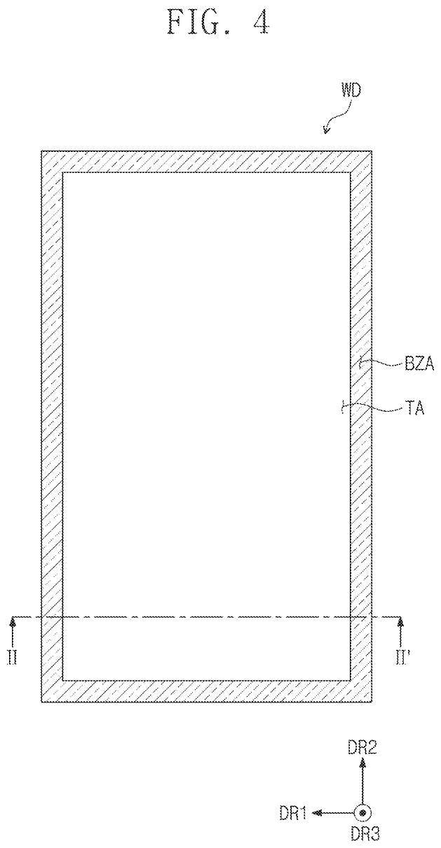

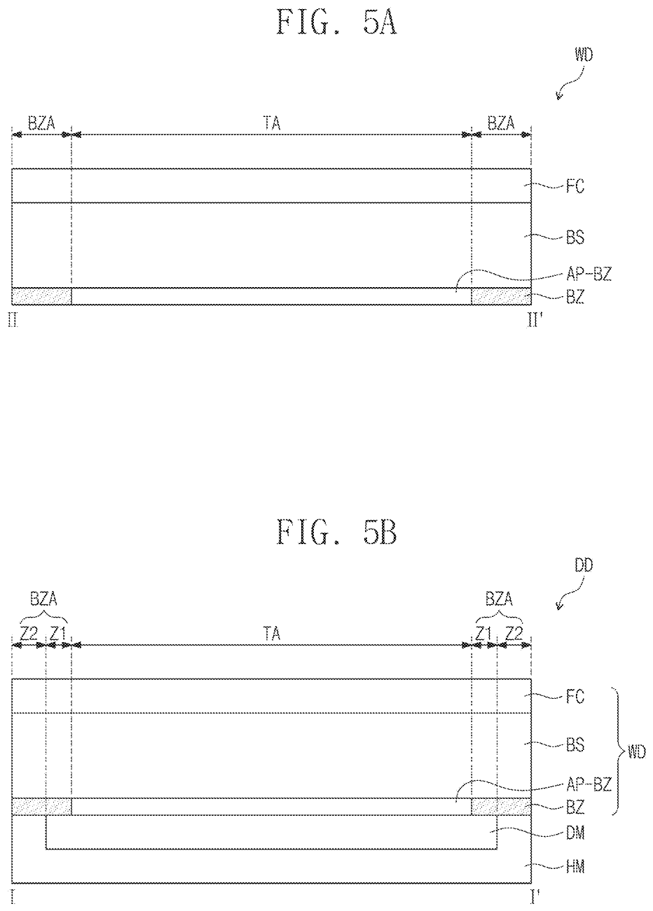

FIG. 4 is a plan view of a window WD according to some embodiments. FIG. 5A is a cross-sectional view of the window WD according to some embodiments. FIG. 5B is a cross-sectional view of the display device DD according to some embodiments. FIG. 5A partially illustrates a cross-section, according to some embodiments, taken along the line II-II′ of FIG. 4. FIG. 5B partially illustrates a cross-section, according to some embodiments, taken along the line I-I′ of FIG. 1.

Referring to FIGS. 4 and 5A, the window WD according to some embodiments may include the bezel region BZA and the transmissive region TA adjacent to the bezel region BZA. The bezel region BZA may have relatively lower transparency than the transmissive region TA. The bezel region BZA may define a shape of the transmissive region TA. The bezel region BZA may be adjacent to the transmissive region TA, and may surround the transmissive region TA.

Referring FIG. 5A, the window WD according to some embodiments may include a base layer BS, a window functional layer FC located on the base layer BS, and a bezel pattern BZ and a lower adhesive layer AP-BZ located on a lower surface of the base layer BS.

The base layer BS may be optically transparent. According to some embodiments, the base layer BS may have a flexible characteristic. For example, the base layer BS may include a synthetic resin such as a polyimide-based resin. Alternatively, the base layer BS may include an ultra-thin glass (UTG). For example, the base layer BS may include glass and have a thickness of about 20 μm to about 80 μm. When the thickness of the base layer BS is less than about 20 μm, the window WD may not ensure sufficient rigidity, and thus be easily damaged by an external impact. When the thickness of the base layer BS is more than about 80 μm, the flexible characteristic of the window WD is deteriorated, and thus the folding and unfolding operations of the display device may not be easily performed.

In the window WD according to some embodiments, the bezel pattern BZ may be located on a lower surface of the base layer BS. The bezel pattern BZ may include at least one of a colored color layer or a black light-shielding layer. The bezel pattern BZ may include a plurality of light-shielding layers as needed. The bezel pattern BZ may be obtained by printing, onto the base layer BS, an ink containing a light-shieling component. The bezel pattern BZ may substantially define the bezel region BZA.

In the window WD according to some embodiments, the lower adhesive layer AP-BZ may be located on the lower surface of the base layer BS. The lower adhesive layer AP-BZ may include an optically clear adhesive (OCA) and/or a pressure sensitive adhesive (PSA). The lower adhesive layer AP-BZ may bond the display module DM (see FIG. 5B) and the base layer (BS). Meanwhile, unlike what is illustrated in FIG. 5A, a portion of the lower adhesive layer AP-BZ may be located below the bezel pattern BZ.

The window functional layer FC may be located on the base layer BS. The window functional layer FC may include at least one of an anti-fingerprint layer, a fragrance-emitting layer, or an anti-reflection layer. The window functional layer FC may include a hard coating layer to protect the window WD from external impact. Examples of the window functional layer FC are not limited thereto, and detailed descriptions related to the window functional layer FC will be described in more detail later.

Referring to FIG. 5B, the window WD according to some embodiments may include a first region Z1 and a second region Z2 adjacent to the first region Z1. The bezel region BZA in the window WD according to some embodiments may include the first region Z1 and the second region Z2. The first region Z1 may be defined as a region, of the bezel region BZA, which a display module DM overlaps, and the second region Z2 may be defined as a region of the bezel region BZA, which a set module HM overlaps.

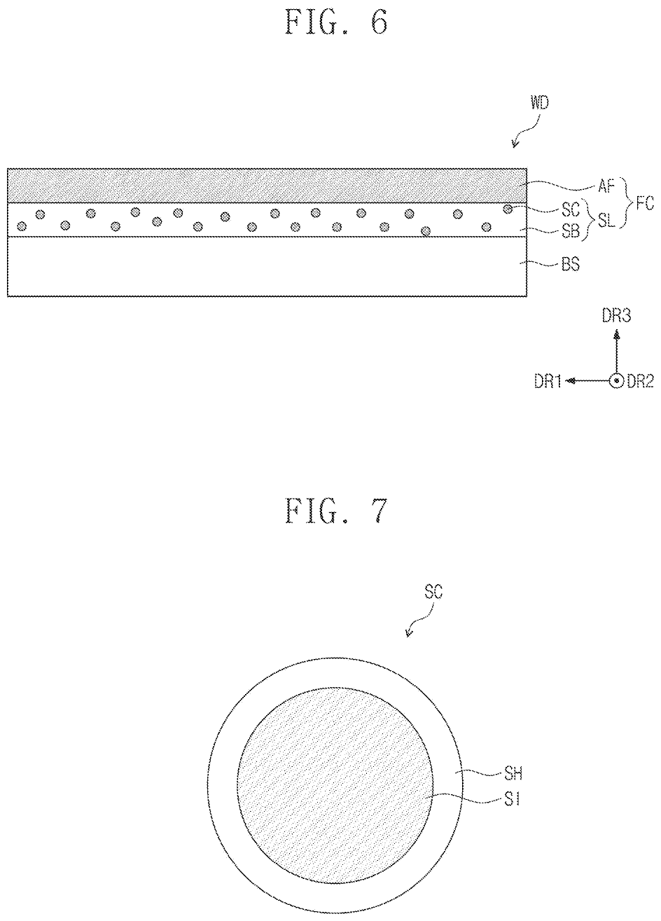

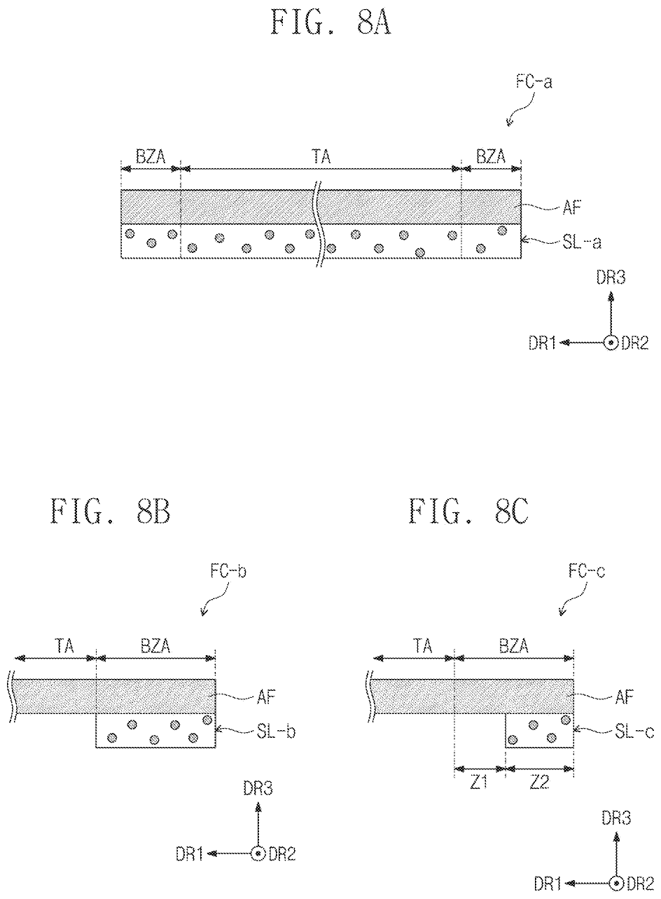

FIG. 6 is a cross-sectional view illustrating some components included in the window WD according to some embodiments of the inventive concept. FIG. 6 illustrates a window functional layer FC according to some embodiments of the inventive concept in more detail. FIG. 7 is a diagram schematically illustrating a structure of a fragrance-emitting capsule FC according to some embodiments of the inventive concept.

Referring to FIG. 6, the window WD according to some embodiments of the inventive concept may include a base layer BS and a window functional layer FC located on the base layer BS. The window functional layer FC according to some embodiments of the inventive concept may include a fragrance-emitting layer SL and an anti-fingerprint layer AF located on the fragrance-emitting layer SL.

The anti-fingerprint layer AF may be located on the fragrance-emitting layer SL. The anti-fingerprint layer AF may have a fingerprint-resistant function, and thus provide a user with a convenience of using the window WD. According to some embodiments, the anti-fingerprint layer AF may be a functional layer in which an anti-fingerprint material is added to a hard coating material. The anti-fingerprint layer AF may include a fluorine-containing compound. For example, the anti-fingerprint layer AF include a perfluoropolyether (PFPE) compound. Alternatively, the anti-fingerprint layer AF may include a perfluoropolyether-containing silane compound. The anti-fingerprint layer AF may have a thickness of about 10 nm to about 40 nm. The anti-fingerprint layer AF may be formed by at least one of an electron-beam (E-beam) deposition process, a thermal deposition process, or a sputtering process.

The fragrance-emitting layer SL may be located between the anti-fingerprint layer AF and the base layer BS. The fragrance-emitting layer SL according to some embodiments may include a base resin SB and a plurality of fragrance-emitting capsules SC dispersed in the base resin SB. As the fragrance-emitting layer SL according to some embodiments includes the fragrance-emitting capsules SC, the fragrance-emitting layer SL may have a fragrance-emitting ability and thus provide fragrance to a user of a display device.

The fragrance-emitting capsule SC may be a capsule that captures specific fragrance-emitting materials in a central portion thereof and surrounds the specific fragrance-emitting materials with wall materials. Accordingly, the fragrance-emitting capsule SC may control a release of the fragrance-emitting materials. The fragrance-emitting capsule SC may be in a form of a micro-capsule or a nano-capsule depending on a diameter. However, the size of the fragrance-emitting capsule SC according to embodiments to the inventive concept is not limited thereto, and the fragrance-emitting capsule SC may have various sizes in consideration of a thickness of the fragrance-emitting layer SL, a thickness of the window WD, and the like. The fragrance-emitting capsule SC may have a spherical shape. However, embodiments of the inventive concept are not limited thereto, and the fragrance-emitting capsule SC may have various shapes as needed.

The fragrance-emitting capsules SC may be dispersed in the base resin SB. The base resin SB may combine the fragrance-emitting capsule SC and the base layer BS. According to some embodiments, the base resin SB may include a melamine resin or an acrylic resin. The base resin SB may include deionized water (DI water).

Referring to FIGS. 6 and 7, a fragrance-emitting capsule SC according to some embodiments of the inventive concept may include a fragrance source SI and a shell SH surrounding the fragrance source SI.

The fragrance capsule SC may include the fragrance source SI containing an aromatic compound. The fragrance-emitting capsule SC may include the shell SH, which is a wall material surrounding and enclosing the fragrance source SI. The fragrance source SI may be released from the shell SH which is a wall material surrounding the fragrance source SI, under a pressure (e.g., a set or predetermined pressure) or at a temperature (e.g., a set or predetermined temperature).

When the pressure (e.g., a set or predetermined pressure) is applied to an upper surface of the window WD by a user or the like, the fragrance source SI of the fragrance capsule SC may be released from the shell SH. Accordingly, the released fragrance source SI may be diffused and reach the upper surface of the window WD. That is, when a user provides a pressure (e.g., a set or predetermined pressure) while using the display device DD (see FIG. 1) including the window WD according to some embodiments, the user may be provided with fragrance by the released fragrance source SI.

Some fragrance-emitting capsules SC of the plurality of fragrance-emitting capsules SC may be in a state in which the fragrance source SI has been released from the shell SH depending on a pressure applied to the upper surface of the window WD. When each of the fragrance-emitting source SI of about 90% or more of the fragrance-emitting capsules SC among the plurality of fragrance-emitting capsules SC has released from each of the shell SH, the fragrance-emitting layer SL according to some embodiments may lose the fragrance-emitting ability.

A duration, from a time when the fragrance-emitting layer SL according to some embodiments is manufactured, to a time when the fragrance source SI of about 90% or more of the fragrance-emitting capsules SC among the plurality of the fragrance-emitting capsules SC is released from the shell SH, may be about one month to about two years. That is, a fragrance-emitting lifespan of the fragrance-emitting layer SL according to some embodiments may be about one month to about two years. However, the fragrance-emitting lifespan according to some embodiments of the inventive concept is not limited thereto, and may be different from the above-mentioned period depending on a material of the fragrance source SI of the fragrance-emitting capsule SC, a degree of use by a user, a layout structure of the fragrance-emitting layer SL, or the like.

The fragrance source SI may include an aromatic compound. The fragrance source SI may include a polymer resin containing an aroma compound. The fragrance source SI may include a specific aromatic compound as needed. For example, the fragrance source SI may include a specific aromatic compound according to a scent desired to be emitted, such as strawberry scent, lavender scent, rose scent, rosemary scent, peppermint scent, and the like.

As described above, the fragrance source SI may include at least any one of terpenes, terpenoids, phenylpropanoid, allyl isothiocyanate, or allicin according to the scent desired to be emitted. However, embodiments of the inventive concept are not limited thereto, the fragrance source SI may include any of an aromatic compound. The shell SH may include a polymer. The shell SH may include at least one of chitosan, cellulose, or melamine. However, a material of the shell SH is not limited thereto.

According to some embodiments, the fragrance-emitting layer SL may be formed on the base layer BS by at least one of a spin coating process, a spray coating process, a deep coating process, an electron-beam (E-beam) deposition process, or a thermal deposition process. According to some embodiments, the fragrance-emitting layer SL may have a thickness of about 1 nm to about 3000 nm. However, the thickness of the fragrance-emitting layer is not limited thereto, and the fragrance-emitting layer SL may have a thickness differing from the abovementioned thickness in consideration of a material of the base layer BS, a size of the fragrance-emitting capsule SC, the number of the fragrance-emitting capsules SC.

According to some embodiments, the window WD may have a refractive index of about 1.2 to about 1.8. The window WD according to some embodiments may have a light transmittance of about 88% to about 100% for light in a wavelength range of about 400 nm to about 700. Accordingly, even when the window functional layer FC according to some embodiments includes the fragrance-emitting layer SL including a plurality of fragrance-emitting capsules SC, the window WD may keep excellent optical properties.

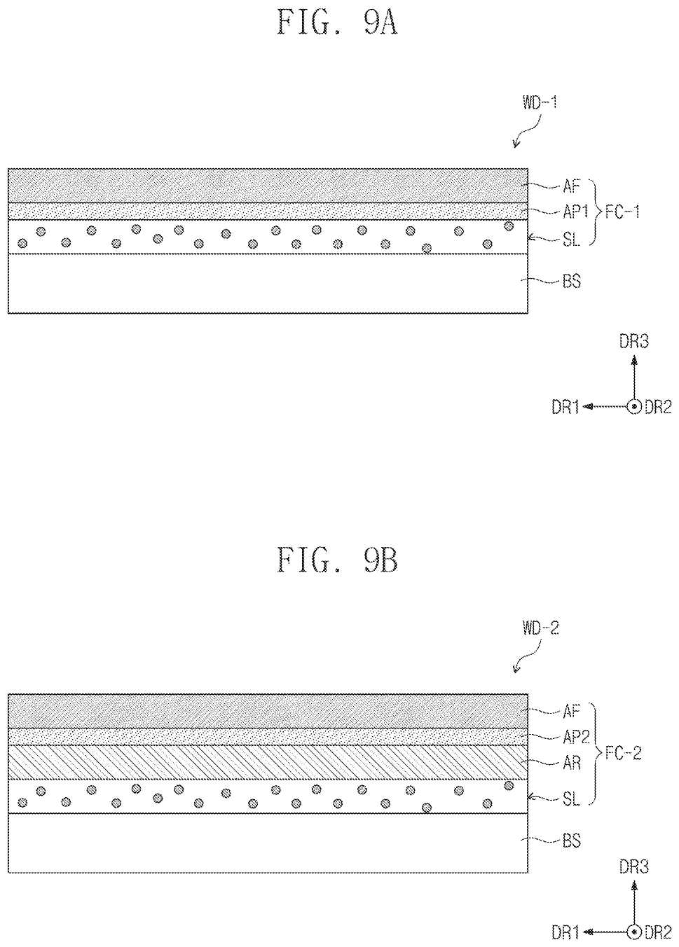

FIGS. 8A through 8C are cross-sectional views illustrating some components included window functional layers FC-a, FC-b, and FC-c respectively, according to some embodiments of the inventive concept. FIGS. 8A through 8C illustrate cross-sectional views illustrating window functional layers FC-a, FC-b, and FC-c respectively, according to regions where the fragrance-emitting layer SL illustrated in FIG. 6 is located.

Referring to FIG. 8A, according to some embodiments, a fragrance-emitting layer SL-a of the window functional layer FC-a may overlap the bezel region BZA and the transmissive region TA. For example, the fragrance-emitting layer SL-a according to some embodiments may entirely overlap the bezel region BZA, and may entirely overlap the transmissive region TA. That is, the fragrance-emitting layer SL may be arranged to entirely overlap a lower surface of the anti-fingerprint layer.

Referring to FIG. 8B, according to some embodiments, a fragrance-emitting layer SL-b of the window functional layer FC-b may overlap only the bezel region BZA. That is, the fragrance-emitting layer SL-b according to some embodiments may not overlap the transmissive layer TA. As the fragrance-emitting layer SL-b does not overlap the transmissive layer TA, the light transmittance of a display device DD (see FIG. 1) according to some embodiments of the inventive concept may increase.

Referring to FIGS. 7 and 8B, because the fragrance-emitting layer SL-b does not overlap the transmissive region TA, the number of times of applying a pressure (e.g., a set or predetermined pressure) to the fragrance-emitting layer SL-b decreases, and thus the frequency at which the fragrance source SI in the plurality of fragrance-emitting capsules SC is released from the shell SH may decrease. Accordingly, compared to the fragrance-emitting layer SL-a (see FIG. 8A) which overlaps both the bezel region BZA and the transmissive region TA, the fragrance-emitting layer SL-b which overlaps the bezel region BZA and does not overlap the transmissive region TA may perform a fragrance-emitting function for a long period of time with low intensity and thus the fragrance-emitting lifespan may be extended.

According to some embodiments, the fragrance-emitting layer SL-b of the window functional layer FC-b may overlap entirely the bezel region BZA. Alternatively, the fragrance-emitting layer SL-b of the window functional layer FC-b according to some embodiments may overlap only a portion of the bezel region BZA. For example, the fragrance-emitting layer SL-b may overlap a region adjacent to a camera hole or the like formed for a camera or a sensor to perform signal transmission. Referring to FIGS. 4 and 8B, the fragrance-emitting layer SL-b according to some embodiments may overlap only the bezel region BZA extending from the transmission region TA in the first direction DR1, and may not overlap the bezel region BZA extending from the transmission region TA in the second direction DR2. Alternatively, the fragrance-emitting region SL-b may overlap only a bezel region BZA extending form the transmission region TA in the first region DR1, may not overlap a bezel region BZA extending from the transmission region TA in a direction opposite to the first direction DR1.

Referring to FIG. 8C, according to some embodiments, a fragrance-emitting layer SL-c of the window functional layer FC-c may overlap only a second region Z2, which is a region, of the bezel regions BZA, which the set module HM (see FIG. 5B) overlaps. The fragrance-emitting layer SL-c according to some embodiments may not overlap a first region Z1, which is a region, of the bezel regions BZA, which the display module DM (see FIG. 5B) overlaps.

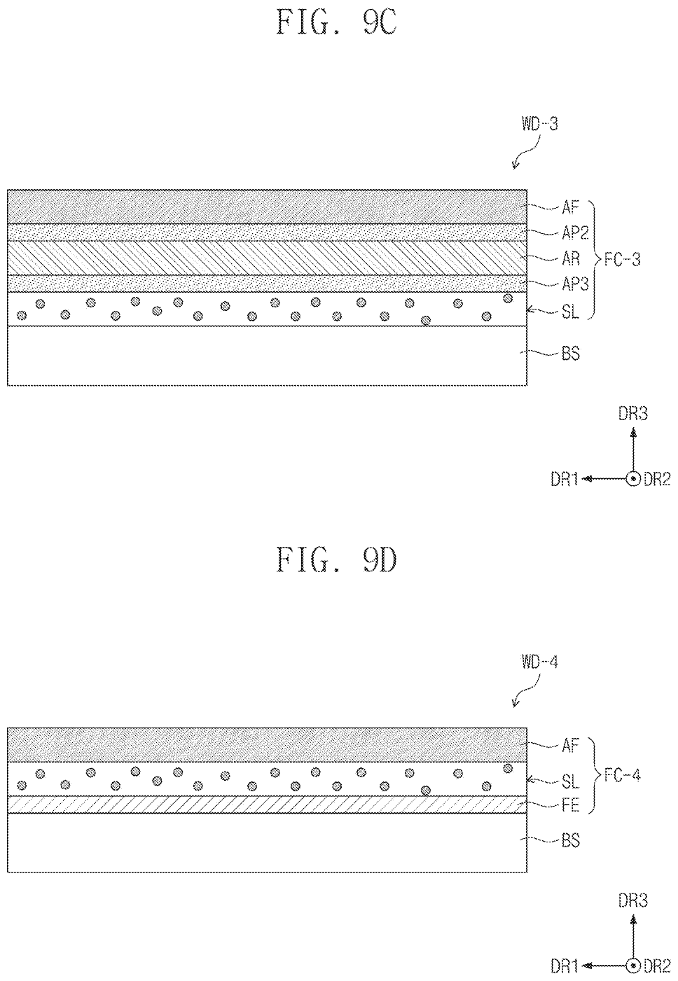

FIGS. 9A through 9D are cross-sectional views illustrating some components included in windows WD-1, WD-2, and WD-3, respectively, according to embodiments of the inventive concept. FIGS. 9A through 9D illustrate window functional layers FC-1, FC-2, FC-3, and FC-4, respectively, according to embodiments of the inventive concept in detail.

Referring to FIG. 9A, a window WD-1 according to some embodiments of the inventive concept may include a base layer BS and a window functional layer FC-1 located on the base layer BS.

The window functional layer FC-1 according to some embodiments of the inventive concept may include a fragrance-emitting layer SL, a first boning layer AP1 located on the fragrance-emitting layer SL, and an anti-fingerprint layer AF located on the first bonding layer AP1.

The window functional layer FC-1 according to some embodiments of the inventive concept may include the first bonding layer AP1 located between the anti-fingerprint layer AF and the fragrance-emitting layer SL. The first bonding layer AP1 may be located between the anti-fingerprint layer AF and the fragrance-emitting layer SL, and bond the anti-fingerprint layer AF and the fragrance-emitting layer SL. The bonding layer AP1 may include silicon or aluminum. For example, the first bonding layer AP1 may include a silicon oxide or an aluminum oxide. According to some embodiments, the first bonding layer AP1 may include a SiO₂ compound, an Al₂O₃ compound, or a combination thereof.

The first bonding layer AP1 may have a thickness of about 1 nm to about 30 nm. The first bonding layer AP1 may have a refractive index of about 1.3 to about 1.6. Accordingly, even when the first bonding layer AP1 of the window WD-1 according to some embodiments is added, the light transmittance or optical properties of the window WD-1 may not be affected.

Referring to FIG. 9B, a window WD-2 according to some embodiments of the inventive concept may include a base layer BS and a window functional layer FC-2 located on the base layer BS. Referring to FIG. 9C, a window WD-3 according to some embodiments of the inventive concept may include a base layer BS and a window functional layer FC-3 located on the base layer BS.

Referring to FIGS. 9B and 9C, window functional layers FC-2 and FC-3 according to some embodiments of the inventive concept may include a fragrance-emitting layer SL, an anti-reflection layer AR located on the fragrance-emitting layer SL, a second bonding layer AP2 located on the anti-reflection layer AR, and an anti-fingerprint layer AF located on the second bonding layer AP2.

Window functional layers FC-2 and FC-3 according to some embodiments of the inventive concept may include a second bonding layer AP2 located between the anti-reflection layer AR and the anti-fingerprint layer AF. The second bonding layer AP2 may be located between the anti-reflection layer AR and the anti-fingerprint layer AF and bond the anti-reflection layer AR and the anti-fingerprint layer AF. The second bonding layer AP2 may have same physical properties or same materials as the above-mentioned first bonding layer AP1 (see FIG. 9A).

Referring to FIG. 9C, the window functional layer FC-3 according to some embodiments of the inventive concept may further include a third bonding layer AP3 located between the anti-reflection layer AR and the fragrance-emitting layer SL. The third bonding layer AP3 may be located between the anti-reflection layer AR and the fragrance-emitting layer SL and bond the anti-reflection layer AR and the fragrance-emitting layer SL. The third bonding layer AP3 may have same physical properties or same materials as the above-mentioned the first bonding layer AP1 (see FIG. 9A).

Unlike what are illustrated in FIGS. 9B and 9C, the second bonding layer AP2 may be omitted, and, in the window functional layers FC-2 and FC-3, the anti-fingerprint layer AF may be directly located on the anti-reflection layer AR. Alternatively, in the window functional layers FC-2 and FC-3, the anti-reflection layer AR may be located on the anti-fingerprint layer AF.

The window-functional layers FC-2 and FC-3 according to some embodiments of the inventive concept may include an anti-reflection layer AR located on the fragrance-emitting layer SL. The anti-reflection layer AR may reduce the reflectance for external light incident from the outside of the windows WD-2 and WD-3. Accordingly, although the fragrance-emitting layer SL is included in the windows WD-2 and WD-3, the light transmittance of the windows WD-2 and WD-3 for light in a wavelength range of about 400 nm to about 700 nm may be maintained at about 88% or more. Because the windows WD-2 and WD-3 according to some embodiments include the fragrance-emitting layer SL and further include the anti-reflection layer AR, the windows WD-2 and WD-3 may have a surface roughness of about 1 μm to about 20 μm. The anti-reflection layer AR may include a high refractive layer and a low refractive layer. For example, the anti-reflection layer AR may have a structure in which a high refractive layer and a low refractive layer are alternately stacked. The anti-reflection layer AR may include Si₃N₄, SiNₓ, SiO₂, Al₂O₃, Nb₂O₅, AlN, SiO, AlOₓN_{y}, SiOₓN_{y}, SiᵤAlᵥOₓN_{y}, MgF₂, MgO, TiO₂, GeO₂, MgAl₂O₄, BaF₂, CaF₂, DyF₃, YbF₃, YF₃, CeF₃, Ta₂O₅, HfO₂, ZrO₂, MoO₃, or any combination thereof.

Referring to FIG. 9D, a window WD-4 according to some embodiments of the inventive concept may include a base layer BS and a window functional layer FC-4 located on the base layer BS.

The window functional layer FC-4 according to some embodiments of the inventive concept may include an optical layer FE, a fragrance-emitting layer SL located on the optical layer FE, and an anti-fingerprint layer AF located on the fragrance-emitting layer SL.

The window functional layer FC-4 according to some embodiments may include an optical layer FE located on the base layer BS. The optical layer FE may be located between the base layer BS and the fragrance-emitting layer SL. The optical layer FE may include irregularities. The irregularities may be defined on an upper surface of the optical layer FE. Accordingly, the external light incident coming from the upper surface of the window WD-4 may be diffusely reflected, and thus the reflectance for the external light may be reduced. Because the window WD-4 according to some embodiments includes the fragrance-emitting layer SL and further includes the optical layer FE, the window WD-4 may have a surface roughness of about 1 μm to about 20 μm. The optical layer FE may be formed by a process of etching the base layer BS. Alternatively, the optical layer FE may be formed by a process of applying beads on the base layer BS.

A window according to some embodiments of the inventive concept may include an anti-fingerprint layer as a window functional layer, and include a fragrance-emitting layer having a fragrance-emitting function below the anti-fingerprint layer, and the fragrance layer includes fragrance-emitting capsules. When a user of a display device according to some embodiments of the inventive concept provides a pressure (e.g., a set or predetermined pressure) to an upper surface of the window while using the display device, the fragrance source in the fragrance-emitting capsules of the display device may be released. Accordingly, the window according to some embodiments may provide fragrance to the user. In a display device according to some embodiments of the inventive concept, the fragrance-emitting layer may be arranged so as to overlap a window front surface. Alternatively, the fragrance-emitting layer may be arranged to overlap only the bezel region and the like as needed. Accordingly, the display device according to some embodiments may maintain a certain light transmittance, extend a fragrance-emitting lifespan, and also provide delicate fragrance to a user.

According to some embodiments of the inventive concept, a window may include a fragrance-emitting layer to provide a fragrance to users, and may perform a relatively excellent transmission function, etc., of the window while including functional layers such as a fragrance-emitting layer and an anti-fingerprint layer and the like. Accordingly, a display device including the window may have relatively improved reliability and aesthetic properties.

Although the embodiments of the inventive concept have been described, it is understood that the inventive concept should not be limited to these embodiments but various changes and modifications can be made by one ordinary skilled in the art within the spirit and scope of embodiments according to the inventive concept as defined in the appended claims, and their equivalents.

What is claimed is:

1. A window comprising:
a base layer; and
a fragrance-emitting layer on the base layer and including a plurality of fragrance-emitting capsules; and
an anti-fingerprint layer on the fragrance-emitting layer.

2. The window of claim 1, wherein a fragrance-emitting capsule from among the fragrance-emitting capsules includes a fragrance source, and a shell surrounding the fragrance source.

3. The window of claim 2, wherein
the fragrance source is configured to be released from the shell by pressure applied to an upper surface of the window, and
a fragrance lifespan being a period during which each of the fragrance source is released from each of the shell in a range of 90% to 100% among the plurality of fragrance-emitting capsules is in a range of one month to two years.

4. The window of claim 2, wherein the fragrance source includes at least one of terpenes, terpenoids, phenylpropanoid, allyl isothiocyanate, or allicin.

5. The window of claim 2, wherein the shell includes at least one of chitosan, cellulose, or melamine.

6. The window of claim 1, wherein the fragrance-emitting layer further includes a base resin in which the fragrance-emitting capsules are dispersed.

7. The window of claim 6, wherein the base resin combines the fragrance-emitting capsule and the base layer.

8. The window of claim 1, further comprising a first bonding layer between the fragrance-emitting layer and the anti-fingerprint layer and configured to bond the fragrance-emitting layer and the anti-fingerprint layer.

9. The window of claim 8, wherein the first bonding layer includes a silicon oxide or an aluminum oxide.

10. The window of claim 1 further comprising an anti-reflection layer between the fragrance-emitting layer and the anti-fingerprint layer.

11. The window of claim 10, further comprising a second bonding layer between the anti-fingerprint layer and the anti-reflection layer and configured to bond the anti-fingerprint layer and the anti-reflection layer.

12. The window of claim 1, further comprising an optical layer between the base layer and the fragrance-emitting layer,
wherein a plurality of irregularities are defined on an upper surface of the optical layer.

13. The window of claim 1, wherein the window has a refractive index in a range of 1.2 to 1.8.

14. The window of claim 1, wherein the window has a surface roughness in a range of 1 μm to 20 μm.

15. The window of claim 1, wherein the window has a light transmittance in a range of 88% to 100% for light in a wavelength range of 400 nm to 700 nm.

16. The window of claim 1, wherein the fragrance-emitting layer has a thickness in a range of 1 nm to 3000 nm.

17. A display device comprising:
a display module; and
a window on the display module,
wherein the window includes:
a base layer on the display module;
a fragrance-emitting layer on the base layer and including a plurality of fragrance-emitting capsules; and
an anti-fingerprint layer on the fragrance-emitting layer.

18. The display of claim 17, wherein the window comprises a bezel region and a transmissive region,
and the fragrance-emitting layer overlaps the bezel region.

19. The display device of claim 18, wherein the fragrance-emitting layer does not overlap the transmissive region.

20. The display device of claim 18, further comprising a set module below the display module,
wherein the bezel region further includes a first region and a second region,
at least a portion of the display module overlaps the first region,
at least a portion of the set module overlaps the second region, and
the fragrance-emitting layer overlaps the second region.
